(19) United States
(12) Patent Application Publication
(10) Pub. No.: US 2007/0225530 A1
(43) Pub. Date: Sep. 27, 2007

(54) High Selectivity Catalysts for the Conversion of Carbon Tetrachloride to Chloroform

(76) Inventors: Michael T. Holbrook, Baton Rouge, LA (US); John D. Myers, Baton Rouge, LA (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 11/579,189

(22) PCT Filed: May 6, 2005
(86) PCT No.: PCT/US05/15815

§ 371 Date: Oct. 31, 2006

Related U.S. Application Data
(60) Provisional application No. 60/571,369, filed on May 14, 2004.

(51) Int. Cl. B01J 23/42; B01J 21/04; B01J 23/46; C07C 17/00; C07C 19/04

(52) U.S. Cl. 570/261; 502/339

(57) ABSTRACT

A process for the hydrodechlorination of carbon tetrachloride to produce chloroform utilizes a supported catalyst having a bimetallic components of platinum and iridium. The bimetallic catalyst may be promoted with small amounts of a third metal, such as tin, titanium, germanium, rhenium, silicon, lead, phosphorus, arsenic, antimony, bismuth or mixtures thereof. By-product production is decreased and duration of catalyst activity is improved by the use of the catalyst of this invention.

### FIELD OF INVENTION

This invention relates to a process for the hydrodechlorination of carbon tetrachloride to produce chloroform and associated byproducts. More particularly, this invention relates to novel catalysts useful for such process having a two-component metal composition. The catalyst may also be promoted with small amounts of metals such as tin, rhenium, germanium, titanium, lead, silicon, phosphorus, arsenic, antimony, or bismuth. The production of undesirable byproducts, such as methane, is significantly reduced by the process and catalyst of the present invention.

### BACKGROUND OF THE INVENTION

Various methods of dehalogenating saturated and unsaturated organic compounds are known. For example, U.S. Pat. No. 3,579,596, issued to Mullin et al. on May 18, 1971, is directed to the vapor-phase hydrodechlorination of carbon tetrachloride and/or chloroform in the presence of a platinum catalyst. U.S. Pat. No. 5,105,032, issued to Holbrook et al. on Apr. 14, 1992, is directed to a vapor-phase process for the hydrodechlorination of carbon tetrachloride to produce chloroform and methylene chloride utilizing a supported platinum catalyst which has been subjected to chloride pretreatment, and, optionally, promoted with small amounts of metals such as tin. The hydrodechlorination of carbon tetrachloride with hydrogen using peripherally deposited platinum on alumina is also discussed by Weiss et al. in Journal of Catalysis 22, 245-254 (1971). However, as discussed by Noelke and Rase in hid. Eng. Chem. Prod. Res. Dev. 18, 325-328 (1979), such processes have been marked with poor selectivity, rapidly declining catalyst activity and short reactor operating cycles. Various treatments have been explored to improve activity and selectivity. These include pretreating catalysts with sulfur and hydrogen.

More recently, several groups have studied the gas phase, platinum catalyzed hydrodechlorination of carbon tetrachloride to chloroform. Yield loss to byproduct methane was high in all cases. A group from Pohang University in South Korea observed methane selectivity of 17 to 67 percent (%) [Bae, J. W., et al., Applied Catalysis A, 240, 129-141 (2003)] and 21 to 32[Bae, J. W., et al., Applied Catalysis A, 217, 79-89 (2001)] as various operational parameters (temperature, pressure, diluent gas) and catalyst parameters (platinum compound, platinum particle size) were varied. A group from Akzo Nobel has received two U.S. patents [U.S. Pat. Nos. 5,721,189 and 5,962,366] on a method of pretreating a platinum on alumina catalyst such that it does not require the chloriding step described in U.S. Pat. No. 5,105,032. This pretreatment apparently results in platinum particles in the 5 to 8 nanometer size range. The bulk of their examples showed the selectivity to byproduct methane to be in the range of 17 to 30%, although one example in the '366 patent showed methane selectivity as low as 8 to 15%. Literature references to this work describe a 2000 hour demonstration of a catalyst pretreated by this method giving 20 to 25% selectivity to methane [Zhang, Z. C., and Beard, B. C., Applied Catalysis A, 174, 33-39 (1998), Applied Catalysis A, 188, 229-240 (1999), Studies in Surface Science and Catalysis, 130, 725-730 (2000)]. The effect on performance of supporting the platinum metal on supports other than alumina has also been studied. Methane selectivity of a platinum on glass [Prati, L., and Rossi, M., Applied Catalysis B, 23, 135-142 (1999)] and on MgO or CeO₂2 [DalSanto, V., et al., Journal of Molecular Catalysis A, 182-183, 157-166 (2002)] was noted as varying from 16 to 38%. A high chloroform selectivity catalyst based on tungsten carbide has been patented [U.S. Pat. No. 5,426,252] and described in the literature [Delannoy, L., et al., Applied Catalysis B, 37, 161-173 (2002)] giving only 1% yield loss to methane. However, this catalyst deactivated rapidly, conversion dropping from 60 to 30% in 20 hours.

Bimetallic catalysts consisting of Group VIII metals (Pt, Ir, Pd, Rh, Ru) with Group 1B metals (Cu, Ag, Au) are claimed in U.S. Pat. No. 5,097,081 to Atochem and in U.S. Pat. No. 5,146,013 to Solvay. However, these catalysts also give high selectivity to byproduct methane, 16 to 39% and 15 to 22%, respectively. Most recently Legawiec-Jarzyna et al. [Applied Catalysis A, General 271 (2004) 61-68] studied bimetallic mixtures of Group VIII metals, specifically platinum and palladium, as catalysts for the carbon tetrachloride to chloroform reaction. Selectivity to chloroform varied monotonically from about 15% (85% selectivity to C1 to C4+hydrocarbons) to about 85% (15% selectivity to C1 and C2 hydrocarbons) as the percentage of Pt in the bimetallic mixture increased from 0 to 100%.

Nevertheless, there is still a need for a process and enabling catalyst for converting carbon tetrachloride to chloroform (and related byproducts), having a high selectivity to chloroform with minimal formation of byproducts and decline in catalyst activity.

### SUMMARY OF THE INVENTION

One aspect of the present invention is a catalyst useful for the hydrodechlorination of carbon tetrachloride to chloroform, said catalyst comprising an alumina support and incorporated therein a bimetallic composition comprising 0.01 to 5 percent by weight based on the total weight of the catalyst of platinum and 0.01 to 15 percent by weight iridium.

Another aspect of the present invention is the catalyst described above which is exposed to a chloride source such as hydrochloric acid.

Yet another aspect of the present invention is the catalyst of the present invention wherein the platinum is distributed on the alumina support between about 0 up to about 2 millimeters depth from the surface and the iridium is distributed between about 0 up to about 2 millimeters from the surface of said support.

A further aspect of the present invention is a catalyst useful for the hydrodechlorination of carbon tetrachloride to chloroform, said catalyst comprising an alumina support and incorporated therein a metallic composition comprising 0.01 and 5 percent by weight of platinum based on the total weight of the catalyst and 0.01 and 15 percent by weight of iridium based on the total weight of the catalyst, and 0 to 1 percent by weight of a third metal based on the total weight of the catalyst.

A final aspect of the present invention is the catalyst of the present invention wherein the platinum and the iridium are distributed on the alumina support as metallic particles with an average size between about 2.5 and about 2000 nanometers.

It is surprising that by the catalyst of this invention, the production of by-products such as methane is significantly reduced; carbon tetrachloride conversion level is maintained relatively constant; and the rate of catalyst deactivation is low.

### DESCRIPTION OF THE DRAWING

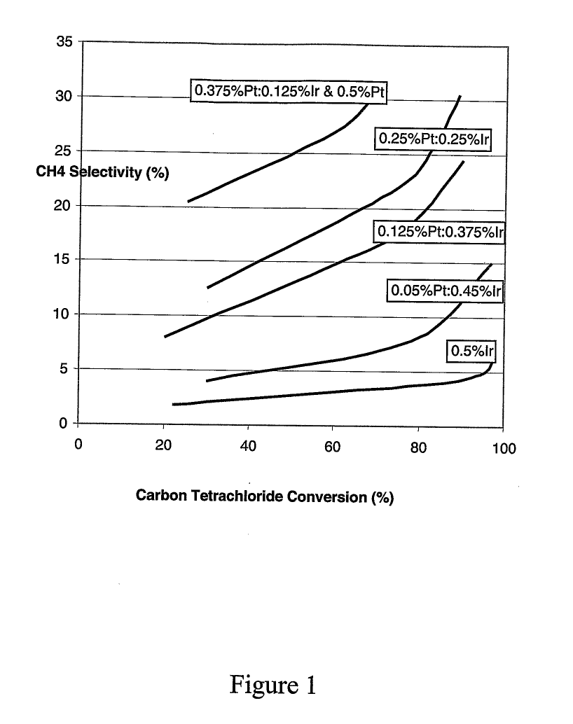

The effects of the platinum:iridium ratio of the catalyst and carbon tetrachloride conversion on selectivity to byproduct methane are shown in FIG. 1. Because the selectivity to byproducts other than methane generally totaled less than 1%, the selectivity to chloroform for the experiments shown in FIG. 1 can be calculated as 100% minus the selectivity to methane. Each curve in FIG. 1 shows results for a particular catalyst. The catalyst giving the lowest selectivity to methane (highest selectivity to chloroform) at a given conversion contained 0.5 wt % Ir. The catalyst compositions giving successively higher selectivities to methane were 0.05% Pt:0.45% Ir, 0.125% Pt:0.375% Ir, 0.25% Pt:0.25% Ir, 0.375% Pt:0.125% Ir, and 0.5% Pt. As indicated above, the last two catalysts gave essentially the same levels of methane selectivity and are both represented by the highest methane selectivity curve on FIG. 1.

### DETAILED DESCRIPTION OF THE ILLUSTRATIVE EMBODIMENTS

The present invention relates to a vapor phase process wherein carbon tetrachloride is contacted with hydrogen in the presence of a bimetallic catalyst of platinum and iridium on alumina under reaction conditions sufficient to form chloroform and trace amounts of other related compounds.

As used herein, the term “selectivity” to an organic product is defmed as the moles of that product exiting the reactor divided by the total number of moles of all organic products (not considering unconverted organic feed) exiting the reactor times 100%. The term “Conversion” is defined as 100% times the difference in the number of moles of organic feed introduced to the reactor and the number of moles of organic feed exiting the reactor, that quantity divided by the number of moles of organic feed. The term “byproducts or related compounds” shall mean any reaction product(s) of such process, other than chloroform, which are co-produced, including, without limitation, methane, methylene chloride, perchloroethylene and hexachloroethane.

The carbon tetrachloride and hydrogen are contacted with catalyst of the present invention at any temperature and pressure at which the desired hydrodechlorination will occur. It is preferred that the temperature is at least about 50° C. and no greater than about 200° C.; more preferred that the temperature is at least about 60° C. and no greater than about 150° C.; and most preferred that the temperature is at least about 70° C. and no greater than about 130° C. The pressure is preferred to be at least about atmospheric and no greater than about 200 psig; more preferred that the pressure is at least about 15 psig and no greater than about 150 psig; and most preferred that the pressure is at least 25 psig and no greater than 100 psig. It will be recognized by one skilled in the art that higher temperatures and pressures are operable in the practice of this invention, but may not be preferred due to economic or other considerations. The process may be conducted in a batch or continuous manner.

Hydrogen and carbon tetrachloride are reacted with the catalyst of this invention to form primarily chloroform and methane. In some preferred embodiments, hydrogen chloride may also be included in the reactant feed. Any amounts of hydrogen, carbon tetrachloride and, optionally, hydrogen chloride which will result in the formation of chloroform and at an acceptable yield are useful in the practice of the present invention. Preferably the mole ratio of hydrogen to carbon tetrachloride in the reactor feed ranges from about 1:1 to about 50:1: more preferably from about 3:1 to about 30:1 and even more preferably from about 6:1 to about 20:1. The presence of hydrogen chloride in the reactant feed most often occurs as a result of recycling unused hydrogen from the product purification section of the process back to the feed to improve the economics of the overall process. The mole ratio of hydrogen chloride to carbon tetrachloride ranges from about 0:1 to about 0.3:1; more preferably from about 0:1 to about 0.1:1. The upper limit on the amount of hydrogen chloride present in the reactant feed is related to catalyst activity. The activity of the catalyst, as determined by carbon tetrachloride conversion, appears to decrease as the amount of hydrogen chloride in the reactant feed increases. However, selectivity to chloroform increases as the amount of hydrogen chloride in the reactant feed increases. Thus, one skilled in the art will recognize that the optimum amount of hydrogen chloride to be included in the reactant feed will be selected to balance conversion of carbon tetrachloride and selectivity to chloroform. In reaction schemes wherein it is feasible to recycle significant amounts of carbon tetrachloride, the selectivity to chloroform obtained by higher amounts of hydrogen chloride might outweigh loss of conversion of carbon tetrachloride.

The bimetallic catalysts of this invention comprise platinum and iridium supported on alumina. The catalyst, in certain embodiments, may also contain a third metal component wherein the third metal comprises tin, titanium, germanium, rhenium, silicon, lead, phosphorus, arsenic, antimony, bismuth, or mixtures thereof. It is most preferred that the catalyst contain platinum and iridium.

The desired amount of platinum and iridium present on the finished catalyst of this invention is defined by the molar ratio of the two metals and by the total metal loading, that is, the sum of the weight percent loading of each metal. The ratio of the metals is defined as the ratio of the number of moles of each metal in the catalyst. Thus, for example, a molar ratio of 25:75 for platinum:iridium would mean that the catalyst would contain 25 atoms of platinum for every 75 atoms of iridium. A total metal loading of 1 wt % at a platinum: iridium molar ratio of 25:75 translates into an individual metal loading of 0.253 wt % platinum and 0.747 wt % iridium, accounting for the difference in molecular weights of the two metals. Thus, the catalyst composition is completely defined by the ratio and total loading of platinum and iridium as further described herein.

The ratio of platinum and iridium according to this invention is best maintained within certain limits. When the platinum: iridium ratio becomes very rich in platinum, the resulting catalyst behavior resembles a pure platinum catalyst with no significant benefit. Specifically, when the platinum: iridium ratio has a platinum content higher than 75:25, the performance of the catalyst is very similar to a pure platinum catalyst with regard to selectivity to the desired product and catalyst deactivation. Thus, there is, for example, no observable benefit at a ratio of 85:15 or 95:5 compared to a pure platinum catalyst. It should be noted that the activity of a 75:25 platinum:iridium catalyst is significantly higher than either pure platinum or pure iridium.

However, the activity of all platinum or platinum/iridium catalysts is so high that further increases do not give a significant economic advantage.

Because of practical considerations, the desired ratio of platinum:iridium is also constrained on the other end of the combinational spectrum, that is at very low platinum:iridium ratios. Selectivity to chloroform from carbon tetrachloride is improved even at a platinum:iridium ratio of 0:100, that is, a pure iridium catalyst. However, a pure iridium catalyst suffers from very rapid loss of activity. Therefore, an acceptable rate of deactivation defines the lower end of the platinum:iridium ratio. It has been found that the presence of even small amounts of platinum in the catalyst can greatly reduce the observed rate of deactivation. The most preferred lower limit of the platinum:iridium ratio is 2.5:97.5. Thus, the preferred platinum:iridium ratio can vary from 2.5:97.5 to 75:25. that is, the platinum portion of the ratio can vary from 2.5% to 75% with a corresponding variation for the iridium such that the percentages for platinum and iridium add up to 100%. There are other factors to consider in determining the total metal loading on the catalyst.

The lowest commercially feasible loading is defmed by the lowest acceptable rate of conversion in a given reactor. The platinum/iridium catalysts of the present invention are very active for the conversion of carbon tetrachloride to chloroform. Consequently, only relatively small amounts of platinum/iridium are required to be in the reactor to render the catalyst commercially viable. The lower limit for total metal loading is estimated to be about 0.01 percent by weight based on the total weight of catalyst in the reactor.

The upper limit for total metal loading is determined by balancing the cost of platinum/iridium catalyst with the desired activity for the resulting catalyst in a given reactor. It is believed that those skilled in the art should be able to define the right balance for the reactor without undue experimentation.

The amount of platinum present in the catalyst is preferably at least about 0.01 percent by weight based on the total weight of the catalyst and no greater than about 5 percent by weight. Preferred ranges are from about 0.05 to about 1 percent by weight.

The amount of iridium present in the catalyst is preferably at least about 0.01 percent by weight based on the total weight of the catalyst and no greater than about 15 percent by weight. Preferred ranges are from about 0.25 to about 5 percent by weight.

The catalyst useful in the present invention is preferably supported. It is preferred that the support be a porous, adsorptive, high-surface area support having a surface area of about 25 to about 500 square meters per gram. Non-limiting examples of suitable support materials include activated carbon, coke or charcoal; silica or silica gel, silicon carbide, clays and silicates including those synthetically prepared and naturally occurring, which may or may not be acid treated, for example attapulgus clay, diatomaceous earth, fuller's earth, kaoline, kieselguhr, etc.; inorganic oxides such as alumina, titanium dioxide, zirconium dioxide, chromium oxide, zinc oxide, magnesia, thoria, boria, silica-alumina, silica-zirconia, silica-magnesia, chromia-alumina, etc.; crystalline zeolitic aluminosilicates; and combinations of one or more elements from one or more of these groups. It is preferred to use alumina supports for the catalysts of the present invention.

Preferred supports have surface areas ranging from about 50 to about 350 square meters per gram, more preferably from about 80 to about 250 square meters per gram. The average pore diameter of the preferred support ranges from about 25 to about 2000 Angstroms, more preferably from about 50 to about 1250 Angstroms. The average diameter of the catalyst is from about. 159 to about 1.27 centimeters (cm) (about 1/16 to about ½ inch).

The platinum, iridium and a third metal may be incorporated into the catalyst support in any suitable manner. Examples of suitable techniques include precipitation, ion-exchange or impregnation. The metals may be incorporated into the support at the same time or may be incorporated separately. In a preferred embodiment, all metal for the catalyst of the present invention is incorporated at the same time.

The method of incorporation into the support is one variable which affects the distribution of the metal on the support. In the catalyst of the present invention, each metal (platinum, iridium and a third metal) may be distributed on the surface of the catalyst support or it may be distributed on or within the support. By distribution of the metal on or within the support, it is meant the distance from the surface of the support that the metal or metals penetrate measured in millimeters. It is preferred that the platinum group metal is distributed between 0 millimeters and no greater than about 2 millimeters from the surface and more preferred that it is distributed no greater than about 1 millimeter from the surface.

Like the platinum and iridium, the third metal may be located on the surface of the support or it may be distributed on or within the support. It is preferred that the third metal is distributed between 0 millimeters and no greater than about 2 millimeters from the surface. It is most preferred the third metal is distributed no greater than about 1 millimeter from the surface.

The depth of penetration of the platinum, iridium, or third metal is not critical to this invention, but serves to optimize the economics of the process. Active metal that is deep inside the support is less efficient at catalyzing the reaction because the reaction rate can be limited by the rate of diffusion of reactants to those active sites.

In addition to platinum, iridium and the third metal, the catalyst may contain other components, such as alkali metal, alkaline metal, halogen, sulfur and other known catalyst modifiers. It is usually the goal when impregnating a precious metal onto a support to make maximum use of that precious metal by dispersing it very finely over the support surface. Thus, only very small metal particles are desired on the catalyst surface. If the metal particles are larger, some of the precious metal is inside these particles, where such precious metal can not affect the desired chemistry and is lost to inefficiency. However, it has been found that very highly dispersing the precious metal in this catalyst can lead to high levels of byproduct formation and to rapid catalyst deactivation. It is preferred that the platinum group metal is distributed on the surface of the catalyst as metallic particles with an average size of between 2.5 nanometers and no greater than about 200 nanometers. It is more preferred that the metallic particles be distributed between 3.0 nanometers and 20 nanometers in size. It is most preferred that these particles be between 4.0 nanometers and about 8.0 nanometer in size. One skilled in the art is aware of various ways to control the size of the metallic particles deposited on the catalyst surface. These include the use of high total metal loading, heating the catalyst in the presence of water or hydrogen or a combination of the two, or by various treatments such as that described in Zhang (U.S. Pat. No. 5,962,366). The approximate average metal particle size can be determined using an electron microscope or by hydrogen or carbon monoxide chemisorption.

Prior to being used in the hydrodechlorination process, the catalyst of the present invention is preferably subjected to a pretreatment comprising treatment with a chloride source. In one preferred embodiment, the catalyst is subjected to a multi-step pretreatment comprising drying the catalyst, reducing the catalyst, and subjecting the catalyst to at least two treatments with a chloride source wherein a later treatment or treatments with the chloride source is conducted at a temperature lower than that used in an earlier treatment.

For example, in one preferred embodiment, the catalyst is subjected to a pre-treatment comprising the following steps:
(1) drying the catalyst under an diluent gas at an elevated temperature:
(2) treating the catalyst with a chloride source selected from the group comprising hydrogen chloride and chlorine at an elevated temperature:
(3) reducing the catalyst: and
(4) treating the catalyst a second time with a chloride source selected from the group comprising hydrogen chloride and chlorine at a temperature less than the temperature used in step (2).

In the drying step, it is preferred that the diluent gas is nitrogen. The temperature is preferably in the range of from about 100° C. to about 500° C. The time required for the drying step is generally in the range of from about one half hour to about forty-eight hours.

In the first chloride treatment, the chloride source with which the catalyst is treated is preferably hydrogen chloride. The temperature in this step of the pre-treatment is preferably in the range of from about 150° C. to about 300° C. The time required for this step is generally in the range of from about one hour to about forty-eight hours.

In the reduction step, the catalyst is reduced using a conventional reducing agent. Examples of suitable reducing agents include hydrogen, hydrazine and formaldehyde. The reducing agent is preferably hydrogen. The temperature in this step of the pre-treatment is preferably in the range of from about 150° C. to about 500° C. The time required for this step is generally in the range of from about two hours to about twenty-four hours. As will be recognized by one skilled in the art, preferred temperatures and times are related so that at higher temperatures, less time will be required and at lower temperatures, more time will be required. The catalyst is cooled after this step, preferably to a temperature in the range of from about 80° C. to about 150° C.

In the second chloride treatment, the chloride source with which the catalyst is treated is preferably hydrogen chloride. The temperature in this step of the pre-treatment is preferably in the range of from about 80° C. to about 150° C. The time required for this step is preferably in the range of from about fifteen minutes to about two hours. The second chloride treatment step should be continued until the desired hydrogen and carbon tetrachloride flows to the reactor are established.

It will be recognized by one skilled in the art that the order of the various parts of the pre-treatment may be varied and in some cases steps will overlap. For example, the catalyst might be treated with a diluent gas and a chloride source at elevated temperatures simultaneously or the treatment with the inert gas and the chloride source may overlap for some period of time.

The following examples are provided to illustrate the invention and should not be interpreted as limiting it in any way. Unless stated otherwise, all parts and percentages are by weight.

Catalyst Preparation

The catalysts useful in this invention may be purchased commercially or made using the general procedure described herein. Samples of active platinum and iridium were obtained from a commercial source as concentrated solutions of the metal chlorides in hydrochloric acid, H₂PtCI₆ and H₂IrCl₆. The correct amounts of these solutions were calculated to give the desired metal loading given the metal concentration in each solution and the total amount of catalyst to be prepared. These amounts were mixed with an appropriate amount of high purity water to obtain the desired total volume of the resulting solution. It was found that relatively uniform coverage of the alumina pellet exterior could be obtained using 1 milliliter (ml) of solution for every 20 grams of alumina. The metal solution was slowly dispensed from a syringe, atomized by an impinging nitrogen stream in the flask, and absorbed by the alumina. The alumina pellets were mixed regularly to give a uniformly edge-loaded catalyst. The catalysts were dried in air overnight at 120° C. They were then reduced under pure hydrogen for 1 hour at 450, 650 or 800° C.

Catalyst Performance Testing

Performance of the various catalyst samples was tested in a reactor system consisting of two independent reactors each with a feed system of Brooks 5850E gas mass flow controllers and a Gilson 305 pump. Carbon tetrachloride was pumped to a preheating oven where the liquid was vaporized and mixed with the pre-heated gas feeds. The evaporated carbon tetrachloride-gas mixture was transferred to a second oven containing a tubular reactor made of 1.27 cm Inconel tubing (0.0889-cm wall thickness) packed with catalyst held in place between plugs of glass wool. Pressure was controlled by varying nitrogen flow through a fixed restriction, created and maintained by using a control valve, downstream of the reactor. Heated transfer lines delivered the reactor effluent to a dedicated Hewlet Packard 5890A gas chromatograph using a 60 meter DB5 capillary column for analysis. A Camile™ (trademark of Camile Products, LLC) computer system controlled the entire process to allow safe, unattended 24-hour operation.

Conversion Reaction

The lab reactor was loaded with 5.0 cc of catalyst—about 4.5 grams for the alumina used.

The loaded reactor was heated to 120° C. at 20 psig under flowing nitrogen and held there for about 30 minutes, followed by heating to 200° C. at 20 psig under an 80:20 mixture of nitrogen and HCl for 1 hour to dry the catalyst. Catalyst reduction was accomplished by flowing an 80:20 mixture of hydrogen and HCl over the catalyst at 200° C. and 20 psig for 1 hour. The reactor was then cooled to 80° C. and the HCl flow was reduced to give a 10:1 hydrogen:HCI molar ratio. The reactor pressure was increased to the desired level (typically 80 psig) during this time. Carbon tetrachloride flow was initiated to give a 12:1 molar hydrogen: carbon tetrachloride feed ratio and a 5 second superficial residence time as calculated for that amount of catalyst. After the carbon tetrachloride flow was well established, the HC1 flow was stopped. In experiments where a significant percentage of the product made was methane, the actual catalyst bed temperature is believed to have been somewhat higher than the oven temperature due to the very exothermic nature of the reaction associated with the formation of methane.

Using the procedure above, a series of platinum:iridium catalysts were made such that the total (platinum+iridium) metal loading was 0.5 wt %, but the ratio of platinum:iridium was varied from pure platinum to pure iridium. Each catalyst was run at 80° C. for at least 50 hours to allow performance to line out. Temperature was then varied to change conversion keeping other parameters constant. A plot of conversion versus selectivity to byproduct methane is shown in FIG. 1 as described above.

### EXAMPLE 1

A catalyst was made having 0.5% iridium on alumina, without any platinum thereon. The average metal particle size as measured by hydrogen chemisorption was 2.3 nanometers. Conversion was varied over the range of about 20% to about 95% by changing temperature. A plot of chloroform selectivity versus carbon tetrachloride conversion is shown in FIG. 1 as the lowest selectivity curve.

### EXAMPLE 2

A catalyst was made having 0.05% platinum and 0.45% iridium on alumina. The average metal particle size as measured by hydrogen chemisorption was 2.7 nanometers. Conversion was varied over the range of about 30% to about 95% by changing temperature. A plot of chloroform selectivity versus carbon tetrachloride conversion is shown in FIG. 1 as the second lowest curve.

### EXAMPLE 3

A catalyst was made having 0.125% platinum and 0.375% iridium on alumina. The average metal particle size as measured by hydrogen chemisorption was 2.9 nanometers. Conversion was varied over the range of about 20% to about 90% by changing temperature. A plot of chloroform 5 selectivity versus carbon tetrachloride conversion is shown in FIG. 1 as the third lowest curve.

### EXAMPLE 4

A catalyst was made having 0.25% platinum and 0.25% iridium on alumina. The average metal particle size as measured by hydrogen chemisorption was 3.5 nanometers. Conversion was varied over the range of about 30% to about 90% by changing temperature. A plot of chloroform selectivity versus carbon tetrachloride conversion is shown in FIG. 1 as the fourth lowest curve.

### EXAMPLE 5

A catalyst was made having 0.3 75% platinum and 0.125% iridium on alumina. The average metal particle size as measured by hydrogen chemisorption was 4.4 nanometers. Conversion was varied over the range of about 20% to about 65% by changing temperature. A plot of chloroform 15 selectivity versus carbon tetrachloride conversion is shown in FIG. 1 as the highest curve.

### EXAMPLE 6

A catalyst was made having 0.5% platinum on alumina, without any iridium thereon. The average metal particle size as measured by hydrogen chemisorption was 5.5 nanometers. Conversion was varied over the range of about 20% to about 65% by changing temperature. A plot 20 of chloroform selectivity versus carbon tetrachloride conversion is shown in FIG. 1 as the highest curve.

### EXAMPLE 7

A catalyst was made having 0.05% platinum and 0.45% iridium on alumina. The average metal particle size as measured by hydrogen chemisorption was 3.2 nanometers. Approximately 450 grams of this catalyst was loaded into a reactor 10 feet long by 1.5 inches in diameter diluted with unburdened alumina. The catalyst was conditioned and run as described above. At 95° C. and 150 psig, the carbon tetrachloride conversion was about 60%, methane selectivity was 3.5%, and the chloroform selectivity was 96%. This performance was stable through the entire run which ended after 330 hours on line.

What is claimed is:

1. A catalyst useful for the gas phase hydrodechlorination of carbon tetrachloride to chloroform, said catalyst comprising an alumina support and incorporated therein a metallic composition comprising 0.01 to 5.0 percent by weight of platinum based on the total weight of the catalyst and 0.01 to 15.0 percent by weight of iridium based on the total weight of the catalyst.

2. The catalyst of claim 1 wherein said metallic composition comprises 0.03 to 1.0 percent by weight of platinum based on the total weight of the catalyst and 0.10 to 3.0 percent by weight of iridium based on the total weight of the catalyst.

3. The catalyst of claim 1 wherein said metallic composition comprises 0.05 percent by weight of platinum based on the total weight of the catalyst and 0.45 percent by weight of iridium based on the total weight of the catalyst.

4. The catalyst of claim 1-3 wherein said catalyst is exposed to a chloride source prior to the use of said catalyst in said hydrodechlorination of carbon tetrachloride to chloroform.

5. The catalyst of claim 4 wherein said chloride source is hydrogen chloride.

6. The catalyst of claim 5 wherein the platinum is distributed on the alumina support between about 0 up to about 2 millimeters from the surface and the iridium is distributed between about 0 up to 2 millimeters from the surface of said support.

7. The catalyst of claim 5 wherein the platinum is distributed on the alumina support between about 0 up to 1 millimeter from the surface and the iridium is distributed between about 0 up to 1 millimeter from the surface of said support.

8. The catalyst of claim 1 wherein said metallic composition comprises 0.01 to 5 percent by weight of platinum based on the total weight of the catalyst and 0.01 to 15 percent by weight of iridium based on the total weight of the catalyst, and 0 to 1 percent by weight of a third metal selected from a group consisting of tin, titanium, germanium, rhenium, silicon, lead, phosphorus, arsenic, antimony, bismuth or mixtures thereof based on the total weight of the catalyst.

9. The catalyst of claim 5 wherein the platinum is distributed on the surface of the catalyst as metallic particles with an average size of between 2.5 nanometers and no greater than about 200 nanometers.

10. The catalyst of claim 5 wherein the platinum is distributed on the surface of the catalyst as metallic particles with an average size of between 4 nanometers and no greater than about 8 nanometers.

11. The process using the catalyst of claim 1 wherein the mole ratio of hydrogen to carbon tetrachloride in the reactor feed ranges from about 6:1 to about 20:1.
